(19) United States
(12) Patent Application Publication
(10) Pub. No.: US 2026/0337712 A1
(43) Pub. Date: Oct. 29, 2026

(54) Method for determining an amount of bainitic ferrite formed during a steel processing operation, associated monitoring or control methods

(71) Applicant: ArcelorMittal, Luxembourg (LU)

(72) Inventors: Nicolas CHARBONNIER, Metz (FR); Ronan JACOLOT, Landonvillers (FR)

(21) Appl. No.: 19/485,852

(22) PCT Filed: Jun. 5, 2024
(86) PCT No.: PCT/IB2024/055497

§ 371 Date: Nov. 19, 2025

(30) Foreign Application Priority Data
Jun. 29, 2023 (WO) PCT/IB2023/056772

(51) Int. Cl. C21D 11/00; G16C 60/00

(52) U.S. Cl. CPC C21D 11/005; C21D 2211/001; C21D 2211/002; C21D 2211/003; C21D 2211/005; G16C 60/00

(57) ABSTRACT

A method for determining an amount of bainitic ferrite formed during a steel processing operation, the method comprising s1) determining a bainitic ferrite growth rate proportional to: exp(−Q*/RT), Q* being an activation energy for an austenite to bainitic ferrite transformation, and proportional to a moderation coefficient which approaches zero when a free enthalpy of a bainitic ferrite phase approaches a free enthalpy of an austenite phase that depends on a Carbon content (C^{γ}) in the austenite phase, s2) determining a cementite growth rate, depending at least on the Carbon content in the austenite phase, s3) determining values at a next time step for bainitic ferrite, austenite and cementite phases fractions (f_{α}, f_{γ}, f_{C}), based on the bainitic ferrite growth rate and on the cementite growth rate, and updating the Carbon content in the austenite phase based on the values of the phases fractions.

The technical field concerns phase transformation determination, for a steel semi-product undergoing a steel processing operation, in particular bainitic ferrite formation prediction. Such a prediction is useful in particular for monitoring, controlling, designing or tuning such a steel processing operation.

### BACKGROUND

Recent developments in steel for automotive and other applications lead to consider bainite microstructures as good candidates to provide an alternative balance of strength and ductility. Being able to determine how much bainite is being formed in a steel undergoing cooling, or more generally undergoing thermal treatment, is thus very useful, to better control the bainite content of the steel finally obtained. Being able to determine the bainite growth rate in a steel is also very useful for the control of a cooling operation itself, or for the control of a thermal treatment. Indeed, as the bainite formation is exothermal, taking into account how much bainite is being formed enables a more accurate temperature control.

### SUMMARY OF THE INVENTION

Regarding bainite formation, different models have been developed in the last years to predict Time-Temperature-Transformation (TTT) curves, or to predict bainite formation kinetics and final fractions. Some of these models perform well for so called ‘carbide free’ grades (for which there is no or little cementite formation due to high Silicon or Aluminium content), but less accurately for grades with a low silicon content. The article “Modelling Simultaneous Formation of Bainitic Ferrite and Carbide in TRIP Steels” by Fateh Fazeli and Matthias Militzer (ISIJ International, 2012, Volume 52, Issue 4, Pages 650-658, Print ISSN 0915-1559) describes a method for predicting fractions of bainite formed during cooling for a high silicon content. Other models predict well bainite formation for lean-silicon steels, where there may be substantial carbide precipitation, but less accurately for carbide free grades (in particular regarding the final bainite fraction).

In this context, there is a need for a method enabling to determine how much bainite is being formed in a steel whose temperature is lowered below the bainite start temperature T_{B}, both for ‘carbide-free’ grade and lean-silicon ones, and with a good comprise between computing time and accuracy.

The invention provides a method for determining an amount of bainitic ferrite formed in a steel semi-product during a steel processing operation, the steel semi-product having a chemical composition CC, the method comprising the following steps, executed by an electronic device:
Acquiring the chemical composition CC of the steel of said steel semi-product,
s1) determining a bainitic ferrite growth rate, as being proportional to:
   a density of bainitic ferrite nucleation centers,
   exp(−Q*/RT) where T is the temperature of the steel, R is the universal gas constant and Q* is an activation energy for an austenite to bainitic ferrite transformation, and to
   a moderation coefficient, which approaches zero when a difference ΔG_{γ→α} between a free enthalpy of a bainitic ferrite phase and a free enthalpy of an austenite phase approaches zero, the enthalpy of the austenite phase depending at least on a Carbon content in the austenite phase,
s2) determining a cementite growth rate, depending at least on the chemical composition CC of the steel and on the Carbon content in the austenite phase,
s3) determining values at a next time step for bainitic ferrite, austenite and cementite phases fractions, based on the bainitic ferrite growth rate and on the cementite growth rate, and determining a value at the next time step for the Carbon content in the austenite phase based on the values of said phases fractions,
   the set of steps s1, s2 and s3 being executed several times successively.

In the computation of the bainitic ferrite formation, the moderation coefficient employed in the claimed method enables to slow down this formation when the free enthalpy in the bainitic ferrite phase approaches the free enthalpy in the austenite phase. This enables to obtain final bainitic ferrite fractions that are in good agreement with experimental results, for ‘carbide-free’ grades. Besides, this moderation coefficient does not modify substantially the dynamic of formation, when the difference between said free enthalpies is substantial, at the beginning of bainite formation. This enables to obtain also good agreements regarding the formation kinetics.

This moderation coefficient mainly reflects a carbon-content driven equilibrium between the bainitic ferrite phase and the austenite phase. For lean-silicon steels, cementite precipitation modifies the carbon content in the austenite phase, and thus modifies the equilibrium in question. Taking into account this carbon-content driven equilibrium (through the moderation coefficient) together with cementite formation (should cementite precipitates) then enables to predict final bainitic ferrite fractions that are in good agreement with measurements, both for carbide-free grades and for grades with lower silicon contents.

In particular, in this method, the value of the Carbon content in the bainitic ferrite phase employed for computing ΔG_{γ→α} may the same as the value of said Carbon content in the austenite phase, for testing how favorable is a transformation from austenite to bainite ferrite, and if not favorable moderating accordingly the bainitic ferrite growth rate.

The invention also provides method for monitoring a steel processing operation during which a steel semi-product (1) is processed in a steel processing installation (4), the method comprising the following steps:
Acquiring at least an initial temperature (Ti) of the steel semi-product at beginning of the steel processing operation, said temperature being measured by a sensor (24) of the steel processing installation,
Determining an amount of bainitic ferrite (f_{α}) formed in the steel semi-product during the steel processing operation, according to the method of any of claims 1 to 10, said method for determining being executed while taking into account that the steel temperature at the beginning of the steel processing operation is the initial temperature (Ti) measured by said sensor.

The invention also provides a method for controlling a steel processing installation, the method comprising the following steps:
Determining a thermal path, to be followed by the steel semi-product during the steel processing operation, in order to obtain a given target property for the steel semi-product at an end of said processing, said thermal path being determined taking into account bainitic ferrite formation during said processing, an amount of bainitic ferrite formed during said processing being determined according to the determining method above,
Controlling actuators of the steel processing installation for achieving the steel processing operation according to the thermal path determined.

The invention also provides electronic device comprising at least a processor and a memory, configured for executing the determining or the monitoring methods described above.

The invention also provides a computer program comprising instructions to be executed on a computer (said computer being connected to a temperature sensor, actuators or a human-computer interface of the steel processing installation, or a production database, when appropriate, depending on the details of the method executed by said computer) to make the computer execute the method as disclosed above.

### BRIEF DESCRIPTION OF THE DRAWINGS

The invention will now be described in detail and illustrated by examples without introducing limitations, with reference to the appended figure:

FIG. 1 schematically represents phases fractions in a steel at an initial and final time of a phase transformation,

FIG. 2 schematically represents phases fractions in a steel at different times of another phase transformation,

FIGS. 3 to 5 represent test results, together with calculations results determined according to the invention, for isothermal transformations, for three Carbide-Free-Bainite exemplary grades,

FIG. 6 represents test results, plotted against calculations results, for some lean-silicon grades,

FIG. 7 represents test results, plotted against calculations results determined according to the invention, for these lean-silicon grades,

FIGS. 8 and 9 gather test results, both for Carbide-Free-Bainite grades and for lean-silicon grades, plotted against calculation results,

FIG. 10 schematically represents steps executed to determine an amount of bainitic ferrite formed during a steel processing operation, according to an embodiment of the invention,

FIG. 11 schematically represents steps executed to determine an amount of bainitic ferrite formed during a steel processing operation, according to another embodiment,

FIG. 12 schematically represents a steel processing installation, to be monitored or controlled using a method according to the invention,

FIGS. 13 and 14 gather production data, for production carried on the steel processing installation of FIG. 12, using either a control method according to the invention, or a control method of the prior art.

### DETAILED DESCRIPTION

### Bainite Formation

In the methods and devices according to the invention, an amount of bainitic ferrite formed in a steel semi-product during a steel processing operation is determined by executing the following steps:
acquiring a chemical composition CC of the steel of said steel semi-product,
s1) determining a bainitic ferrite growth rate, as being proportional to:
   a density of bainitic ferrite nucleation centers n_{nucl,α}, to
   exp(−Q*/RT) where T is the temperature of the steel, R is the universal gas constant and Q* is an activation energy for an austenite to bainitic ferrite transformation, and to
   a moderation coefficient C_{mod}, which approaches zero when a free enthalpy of a bainitic ferrite phase α approaches a free enthalpy of an austenite phase γ, the enthalpy of the austenite phase depending at least on a Carbon content C^{γ} in the austenite phase γ,
s2) determining a cementite growth rate, depending at least on the chemical composition CC of the steel and on the Carbon content C^{γ} in the austenite phase,
s3) determining values at a next time step for bainitic ferrite, austenite and cementite phases fractions f_{α}, f_{γ} and f_{C}, based on the bainitic ferrite growth rate and on the cementite growth rate, and determining a value at the next time step for the Carbon content in the austenite phase CY, based on the values of the phases fractions f_{α}, f_{γ}, f_{C}.

The set of steps s1, s2 and s3 is executed several times successively, to determine gradually the temporal evolution of the bainitic ferrite content in said steel, during the steel processing operation in question, while taking into account, at each time step, possible Carbon redistribution among the three possible phases mentioned above (through the calculation of the updated value, at each time step, of the Carbon content in the austenite phase C^{γ}), and the influence of this redistribution on the bainitic ferrite and cementite growth rates.

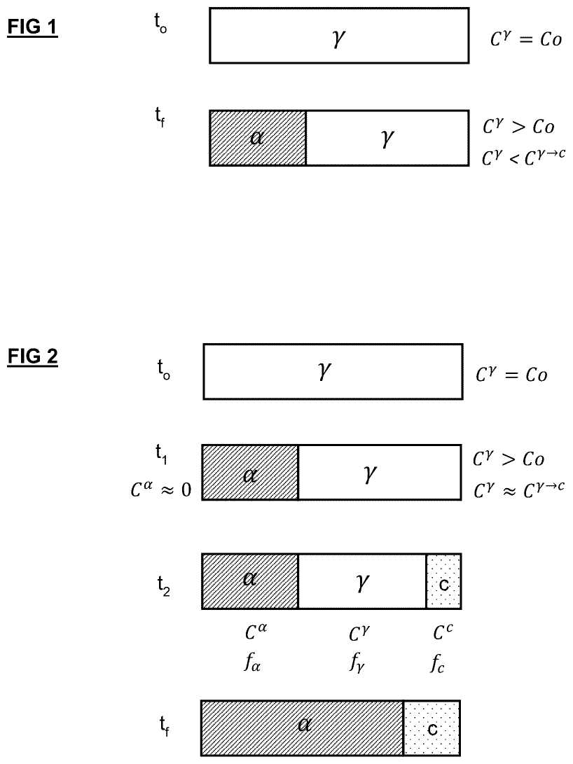

FIG. 1 represents schematically phases fractions in a steel at an initial time tₒ and final time t_{f} of a phase transformation, occurring at a temperature below the Bainite start temperature T_{B} (and above the Martensite start temperature T_{M}), for a steel with a high Silicon content (for instance 1.5 weight % or above), of the CFB type (Carbide-Free Bainite type). At the initial time tₒ, it is considered, in this simplified example, that austenite only is present, with an initial Carbon content in the austenite phase C^{γ}=Cₒ. Some bainitic ferrite then forms, until the transformation stops (because an equilibrium is reached, in this case), at time t_{f}. During this transformation, the Carbon is redistributed: it is rejected from the bainitic ferrite phase α (where the Carbon content C^{α} is negligible compared to the carbon content C^{γ} in the austenite phase) into the austenite phase γ. The Carbon content in the austenite phase, C^{γ}, thus gradually increases.

When computing the bainitic ferrite growth rate as described above, the moderation coefficient C_{mod} enables to slow down the bainitic ferrite formation when the Carbon content in the austenite phase increases, in the course of the transformation, and enables then to obtain final fractions of non-transformed austenite (that are not null, for CFB grades), that are in good agreement with experimental results.

The difference between the free enthalpy of a bainitic ferrite phase and the free enthalpy of the austenite phase is noted as ΔG_{γ→α}. It is a free enthalpy change for diffusion less transformation from austenite to bainitic ferrite. Here, the value of the Carbon content in the bainitic ferrite phase, employed for computing ΔG_{γ→α}, is the same as the value of the Carbon content in the austenite phase, C^{γ}. In other words, ΔG_{γ→α} is the difference between the free energy of the austenite phase, and a free energy of a bainitic ferrite phase that would result from a phase transformation of this austenite phase into bainitic ferrite with no variation of the Carbon content during the transformation itself, to evaluate if the transformation can take place or not.

Here, the moderation coefficient C_{mod} is approximately proportional to |ΔG_{γ→α}|/RT when |ΔG_{γ→α}|/RT approaches zero (for instance, proportional or even equal to ΔG_{γ→α}|/RT, within 5% or less when ΔG_{γ→α}|/RT is smaller than 0.2). This enables to slow down substantially the bainitic ferrite formation when ΔG_{γ→α} approaches zero. Besides, the moderation coefficient C_{mod} is bound, limited to a constant value when |ΔG_{γ→α}|/RT has a high value (eg: higher than 2). This prevents the moderation coefficient from modifying the bainite formation kinetics, at the beginning of the transformation, and enables obtaining good agreement with experimental results regarding the formation kinetics.

To fulfill these criteria, the moderation coefficient C_{mod} is for instance calculated as being equal to tanh(|ΔG_{γ→α}|/RT).

FIG. 2 represents schematically phases fractions in a steel, at an initial time tₒ, a final time t_{f} and two intermediary times t₁ and t₂ of a phase transformation (with t_{f}>t₂>t₁>tₒ). This transformation occurs at a temperature below the Bainite start temperature T_{B} (and above the Martensite start temperature T_{M}), for a steel with a very low Silicon content (for instance less than 0.1 weight %), in which cementite (carbide) precipitation is likely. At the initial time tₒ, only the austenite phase γ is present, with an initial Carbon content C^{γ}=Cₒ. Some bainitic ferrite α then forms and some carbon is thus rejected in the austenite phase where the carbon content C^{γ} increases until this carbon content reaches (at time t₁) a Carbon content threshold C^{γ→c} for cementite precipitation. The cementite phase C then grows up (time t₂). As the carbon content C^{c} in the cementite phase is high (around 6.67 weight %), cementite formation has a pumping action regarding the carbon content C^{γ}, and tends to drastically reduce the carbon content C^{γ} in the austenite phase. So, as C^{γ} remains low, the moderation coefficient C_{mod} remains nonzero, non-negligible, and the austenite to bainitic ferrite transformation continues until no more austenite is present (at time t_{f}).

In the method presented above, taking into account that cementite may form during the steel processing operation, depending on the chemical composition CC of the steel, the cementite formation then pumping carbon from the austenite phase, enables to predict adequate final bainitic ferrite fractions, both for carbide-free grades and for grades with low silicon and aluminum contents (for which the final, remaining austenite phase fraction is usually very low, or even null).

In step s2, the cementite growth rate may remain equal to zero all along the steel processing operation, depending on the chemical composition CC of the steel (in particular for chemical compositions with high Silicon or Aluminum content). Or, on the contrary, it may become non-null during the transformation, for chemical compositions with low Silicon and Aluminum content. In particular, the cementite growth rate may be kept equal to zero as long as the Carbon content in the austenite phase, C^{γ}, remains below the Carbon content threshold for cementite precipitation, C^{γ→c}, above mentioned.

Bainite is a microstructure comprising of very thin ferrite plates, possibly separated by retained austenite. It forms in austenite. The ferrite of these thin ferrite plates is designated as bainitic ferrite (or more simply as bainite), in this document. When cementite forms, it is assumed here that it forms in the austenite (and then has the carbon-pumping action, with respect to the austenite, as above described with reference to FIG. 2).

The steel processing operation, for which the amount of bainitic ferrite is determined, is a processing operation for which bainite is likely to form. It is typically a processing operation:
for which the steel is, for some time, at high temperature, typically above AC3 temperature, and then contains as substantial fraction of austenite, for instance more than 50% wt or even more than 70% wt of austenite, the rest being mainly ferrite,
or for which the steel initially contains such a substantial fraction of austenite (the rest being mainly ferrite).

Then, during this processing operation, the temperature of the steel is decreased and becomes lower than the Bainite start temperature T_{B}. Bainite then forms, its kinetics of formation being determined as above explained.

The gradual calculation of the temporal evolution of the phases fractions f_{α}, f_{γ}, f_{C} starts with initial conditions that are typically f_{γ}(t=0)=f_{γo}, f_{α}(t=0)=0 and f_{C} (t=0)=0, with f_{γo}=1 if the steel initially contains 100% austenite (and no ferrite). This gradual, iterative calculation may be carried on until an end point of the transformation. This end point may correspond to a situation where said phases fractions reach constant, steady-state values (i.e.: a ‘statis’, corresponding to the final time t_{f} of FIGS. 1 and 2), or to an instant the steel processing operation ends (output of the steel semi-product from a steel processing installation where it is processed, for instance).

The steel processing operation in question may be a hot rolling operation, including a steel cooling operation achieved on a Run Out Table, for instance.

The steel semi-product can be a steel strip or sheet, a slab, a billet, a broom, an ingot, a bar, a beam, a tube or a wire. More generally, the steel semi-product is an intermediary source-product, destinated, subsequently, to manufacture a part, a good or a construction.

The chemical composition CC of the steel, taken into account to determine the amount of bainitic ferrite being formed, may be specified by the weight % of the different alloy elements present in said steel, for instance the weight % of C, Mn, Si, Al, Cr, Cu, Mo, Ni. These alloy elements contents are the initial contents in the steel, at the beginning of the steel processing operation. The carbon content, defining said chemical composition CC, may be, more specifically, an initial carbon content in the austenite. It is Co.

If a ferritic transformation occurred, before the bainitic transformation herein modelized starts, the ferrite fraction f_{F} is nonzero, and the initial austenite fraction f_{γo} is below 1. In such a case, the initial carbon content Cₒ, in the austenite phase, is calculated as being equal to Cᵢₙᵢ/f_{γo}, where Cᵢₙᵢ is the carbon content in the steel before the ferritic transformation occurred (in other words, Cᵢₙᵢ corresponds to the average, global carbon content of the steel; to its global chemistry).

The computation steps, described below in more detail, are adequate to compute an amount of bainitic ferrite (in other words, an amount of bainite) formed during a steel processing operation. They can be combined with additional steps to further take into account the possible formation of other types of phases.

In the instant method for determining the amount of bainitic ferrite being formed during such a steel processing operation, the carbon content, either in the austenite, bainitic ferrite, or cementite phase, may be expressed as a weight proportion (weight %) in said phase, as a mass concentration, as a molar concentration, or any other quantity representative of the density of carbon in said phase. Regarding the phase fractions mentioned above, they are volume fractions, here.

### Step s1: Determination of the Bainitic Ferrite Growth Rate

In the method here described, the bainitic ferrite growth rate is computed according to equation eqn 1 below:

$$\frac{d {f}_{α}}{d t} = v \cdot {n}_{n u c l , α} \cdot {f}_{γ} \cdot ( 1 + λ {f}_{α}^{′} ) \cdot exp ( - {Q}^{*} / RT ) \cdot {C}_{m o d} \qquad ( eqn 1 )$$

where
   f_{α} is the volume fraction of bainitic ferrite in the steel,
   v=kT/h=10¹³ s⁻¹ (k being the Boltzmann constant, and h being the Planck constant),
   λ is an auto-catalysis coefficient proportional to a dimension d_{γ} of austenite grains, and
   f′_{α} is equal to f_{α}, or possibly to f_{α}+f_{F}.

The term f_{γ} (for instance equal to (1−f_{α}) in the absence of cementite and in the absence of ferrite—that is if f_{F}=0) reflects that the number of possible nucleation sites decreases gradually as the amount of austenite decreases, when bainitic ferrite forms.

The auto-catalysis coefficient λ is expressed as λ=α·d_{γ}. For the steel grades considered here (whose alloys elements contents are comprised in the ranges specified further below), a value of ‘a’ from 0.1 μm⁻¹ to 0.8 μm⁻¹, or even from 0.1 to 0.5 μm⁻¹ is adequate.

In the instant model, the density of bainitic ferrite nucleation centers n_{nucl,α} is computed according to the following equation:

n_{nucl,α}=α^{b}·(T_{B}−T)

where α^{b} is a quantity related to a density of defects at grains boundaries, favourable to bainitic ferrite growth. α^{b} depends on the chemical composition CC of the steel, and on the austenite grains dimension d_{γ}, but does not depend on the temperature T, nor on the instant Carbon content C^{γ} in the austenite phase. α^{b} may more specifically be determined according to the equation below (where d_{γ} is expressed in meters):

α^{b}=α^{m}·Kst/d_{γ}

where α^{m} is a rate parameter for martensite formation, depending on the steel chemical composition CC and where Kst is a constant coefficient, from 1·10⁻⁹ to 100·10⁻⁹ meters. α^{m} may more specifically be determined according to the following equation, where α^{m} is expressed in K⁻¹:

α^{m}=(27.2−0.14x_{Mn}−0.08x_{Ni}−0.11x_{Cr}−0.05x_{Mo}+0.21x_{Si}−19.8[1−exp(−1.56Cₒ)])/1000

where x_{Mn}, x_{Ni}, x_{Cr}, x_{Mo}, x_{Si} and Cₒ are, respectively, the Manganese, Nickel, Chromium, Molybdenum, Silicon, and Carbon contents for said chemical composition CC, expressed in weight %.

Regarding the activation energy Q* for the austenite to bainitic ferrite transformation, it is calculated here as being equal, or substantially equal to Qₒ+K₁·|ΔG_{γ→α}|, and where Qₒ and K₁ may depend on the chemical composition CC of the steel but remain constant when the Carbon content C^{γ} in the austenite phase varies with time, during the phase transformation of the steel. By substantially equal it is meant equal within 10%, or even within 5% or 3%. The inventors have noticed that this way to compute Q* leads to a good agreement between the model predictions and experimental observations, in terms of kinetics and final phases fractions. Besides, this formulation has the advantage to isolate a part of Q* that varies with C^{γ} and has thus to be recalculated at each time step (namely ΔG_{γ→α}), from a part of Q* independent of C^{γ} (namely Qₒ). Besides, the term varying with C^{γ} is the same as the one in the moderation coefficient C_{mod}, and has thus to be calculated just once when determining the bainitic ferrite growth rate, thanks to this particular way to express Q*. In practice, the value of Qₒ is typically comprised between 150 and 200 kJ/mol. And the value of K1 is from 3 to 10, or even from 5 to 7.

Quantity Qₒ depends on the chemical composition CC of the steel. The inventors have observed that the dependence of Qₒ with the chemical composition CC can be taken into account conveniently by computing Qₒ as being equal, or substantially equal to A+B·(T−T_{M}), A and B being two constants and T_{M} being the Martensite start temperature. In other words, the inventors have noticed that this expression fits well the values of Qₒ. In this expression, the dependency of Qₒ with the chemical composition CC is reflected by the dependency of T_{M} with the chemical composition CC, dependency for which numerous data and studies are available. In practice, the value of constant A is typically comprised between 160 et 200 kJ/mol while the value of B is typically comprised between 100 and 150 J/(mol·K). For instance, the tests presented below, with reference to the figures, are carried on with λ=177 kJ/mol, B=120 J/(mol·K) and K1=6.

For a given chemical composition, the value of the Martensite start temperature T_{M} can be experimentally determined by dilatometry. It can also be found in data from the literature. It can also be determined using the following equation (valid for the alloy elements content ranges defined further below), where T_{M} is in Celsius degrees (°C):

T_{M}=565−31·x_{Mn}−13·x_{Si}−10·x_{Cr}−18·x_{Ni}−12·x_{Mo}−600·(1−exp(−0.96·x_{C}))

Similarly, the value of the Bainite start temperature T_{B} can be experimentally determined by dilatometry and microstructural analysis, or found in data from the literature. It can also be determined using the following equation (valid for the alloy elements content ranges defined further below), where T_{B} is in °C:

T_{B}=839−86·x_{Mn}−23·x_{Si}−67·x_{Cr}−33·x_{Ni}−75·x_{Mo}−270·(1−exp(−1.33·Cₒ))

where x_{Mn}, x_{Si}, x_{Cr}, x_{Ni}, x_{Mo} and Cₒ, are, respectively, the Manganese, Silicon, Chromium, Nickel, Molybdenum and Carbon contents corresponding to the chemical composition CC of the steel, expressed in weight %.

Regarding the austenite grains dimension d_{γ}, taken into account to compute the bainite growth rate, it may be measured, on a sample taken from the steel semi-product before its processing, or taken from another semi-product of a same group of semi-products (produced during a same production campaign). Alternatively, the grains dimension d_{γ} may also be calculated, based on the characteristics of previous processing underwent by the steel semi-product (underwent before the instant steel processing operation). It could also be determined, based on these previous process characteristics, using a look-up-table. Anyhow, the grains dimension d_{γ} is defined, as usual in this technical field, as a kind of average diameter of the grains. From a geometric point of view, it is determined using the so-called intercepts method: in an image of the sample, a number of vertical and horizontal lines are drawn; then, each time a line passes over a grain boundary, the dimension (width) of the grain along this line is determined. A vertical and a horizontal average are thus obtained. The average of these two averages is the grain dimension d_{γ}.

Taking into account the expressions employed here for n_{nucl,α} and Q*, the bainite growth rate reads (eqn 1a):

$$\frac{d {f}_{α}}{d t} = v \cdot {α}^{b} \cdot ( {T}_{B} - T ) {f}_{γ} ( 1 + λ {f}_{α}^{′} ) \cdot exp ( - {Q}_{0} / RT ) \cdot exp ( - {K}_{1} \cdot \vert Δ {G}_{γ \to α} \vert / RT ) \cdot tanh ( \vert Δ {G}_{γ \to α} \vert / RT ) \qquad ( eqn 1 a )$$

In equation eqn 1a, the quantities that vary, or that may vary during the transformation, and whose values are updated at each time step, are:
the bainitic ferrite phase fraction f_{α},
the temperature T (which may change during the transformation), and
ΔG_{γ→α}, whose variations are mainly caused by the variation of the Carbon content in the austenite phase, C^{γ}.

Remarkably, the other parameters of eqn 1a remain constant during the transformation.

Regarding ΔG_{γ→α} (which varies with variation with C^{γ}), it is computed by calculating:
a free enthalpy for a single austenitic phase, given the carbon content C^{γ} in that phase, and for other elements contents, like x_{Mn}, x_{Si}, x_{Mo}, . . . , given the chemical composition CC of the steel),
a free enthalpy for a single ferritic phase, given the elements contents (x_{Mn}, x_{Si}, x_{Mo}, . . . ) specified by the chemical composition CC of the steel, and
subtracting one from the other.

In the embodiment described here (which corresponds to the test results presented below), the value of the Carbon content in this (bainitic) ferritic phase, employed for computing ΔG_{γ→α}, is the same as the value of the Carbon content in the austenitic phase, C^{γ}. In other words, the free enthalpy for the ferritic phase in question is computed assuming that:
the elements contents (x_{Mn}, x_{Si}, x_{Mo}, . . . ) are the same as in the austenitic phase, and
that the Carbon content is also the same as in the in the austenitic phase.

ΔG_{γ→α} is thus the enthalpy difference between the austenite phase γ and a (bainitic) ferrite phase that would result from the transformation of this austenite phase into a ferrite phase while keeping the same Carbon content and keeping the same additional elements contents (i.e.: while keeping the steel composition constant); in other words, ΔG_{γ→α} is the enthalpy difference for a so-called “displacive” transformation from austenite to bainitic ferrite. This is different from an enthalpy difference for a so-called “para-equilibrium” phase transformation, for which the free enthalpy for the austenite phase and the free enthalpy for the bainitic ferrite phase are computed using respectively a Carbon content for the austenite phase, and another Carbon content for the bainitic ferrite phase, different from each other (and which are more precisely the equilibrium Carbon contents for these two phases).

It is noted that it is just for the computation of ΔG_{γ→α} (for computing the transformation kinetics) that the same value of Carbon content is taken both for the austenite phase and for bainitic ferrite phase. In the bainitic phase α actually formed, the Carbon content C^{α} is different from the Carbon content C^{γ} in the austenite phase γ; the respective values of C^{γ} and C^{α} are computed and updated as below explained when describing step s3.

These values of free enthalpy may be calculated using commercial thermodynamic calculations software, like ThermoCalc software, for instance.

### Step s2: Determination of the Cementite Growth Rate

As above explained, the cementite growth rate is kept equal to zero as long as the Carbon content in the austenite phase, C^{γ}, is below the Carbon content threshold for cementite precipitation, C^{γ→c}.

Once the Carbon content in the austenite phase C^{γ} has passed the threshold in question, cementite forms from the austenite.

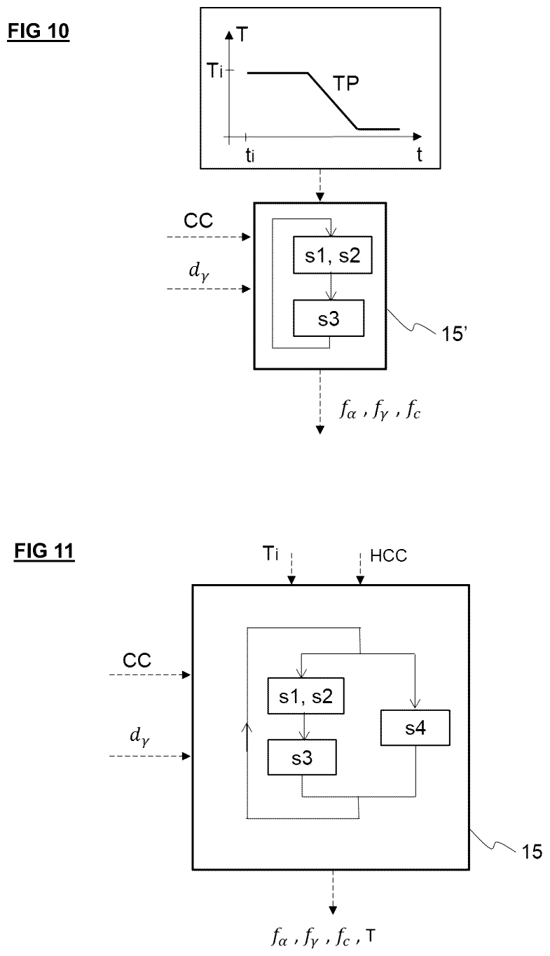

The carbon content threshold for cementite precipitation, C^{γ→c}, can be determined according to the following article: “Effect of partitioning of Mn and Si on the growth kinetics of cementite in tempered Fe—0.6 mass % C martensite”, by Miyamoto, J. C. Oh, K. Hono, T. Furuhara, and T. Maki, Acta Materialia, Volume 55, Issue 15, 2007, Pages 5027-5038. In particular, table 5 of this article provides values for enthalpies of formation of M₃C compounds (M being an alloy element present in the steel, like Si, or Cr), from which the free enthalpy ΔrG of formation of cementite in austenite can be calculated, for the steel considered (depending on the chemical composition CC, and with a preequilibrium hypothesis), which enables to determine the carbon content threshold for cementite precipitation, C^{γ→c}. As a matter of example, FIG. 10 of this article provides limits (“PE” line in FIG. 10 a and b, for “ParaEquilibrium”, which is assumed here), in terms of carbon concentration, for the existence of the cementite phase (θ), as a function of the Si or Mn content.

The cementite growth rate can be calculated according to the following article: “Modelling upper and lower bainite transformation in steels” by M. Azuma, N. Fujita, M. Takahashi, T. Lung, ISIJ International, vol. 45 (2005), n°2, pp 221−228, in particular as described in section 3.3 and 3.4 of that article. The information regarding cementite nuclei growth, presented in the preceding article by Miyamoto (see table 4, and eqn 2 to 4 of Miyamoto's article) can also be used for cementite growth calculation.

### Step s3: Determination of the Carbon Content in the Austenite Phase

In step s3, the Carbon content in the austenite phase, C^{γ}, is computed while neglecting a Carbon content in the bainitic ferrite phase C^{α} with respect to C^{γ}, and with a constant carbon content in the cementite phase C^{c}, equal to 6.67 weight %.

Taking into account the conservation of the overall quantity of Carbon initially present, C^{γ} is then computed according to the equation below:

$${C}^{γ} = \frac{{C}_{o} - {f}_{α} \cdot {C}^{α} - {f}_{c} \cdot {C}^{c}}{{f}_{γ}} ≅ \frac{{C}_{o} - {f}_{c} \cdot {C}^{c}}{{f}_{γ}}$$

with f_{γ}=1−f_{α}−f_{c}, or possibly=f_{γ}(tₒ)−f_{α}−f_{c}.

The validity domains, for the above calculation method (both for the bainitic ferrite and cementite formation) are the followings, expressed in weight %:
Cₒ≤0.5 or even ≤0.3, ≤0.25, or ≤0.2
x_{Mn}≤3 or even ≤2.5, or ≤2
x_{Si}≤2, or even 1.5
x_{Al}≤1.5
x_{Cr}≤4, or even ≤1, or ≤0.8
x_{Mo}≤0.8 or even ≤0.5
x_{Ni}≤2.

The numerical formulas above, for instance the formulas enabling to compute T_{B} or T_{M}, provide valid predictions, in the ranges mentioned above. And the overall method, for determining the amount of bainite formed, leads to reasonably accurate predictions, within these ranges, as illustrated by the exemplary experimental test results presented below.

### Exemplary Test Results

### Carbide Free Bainite Steel Grades

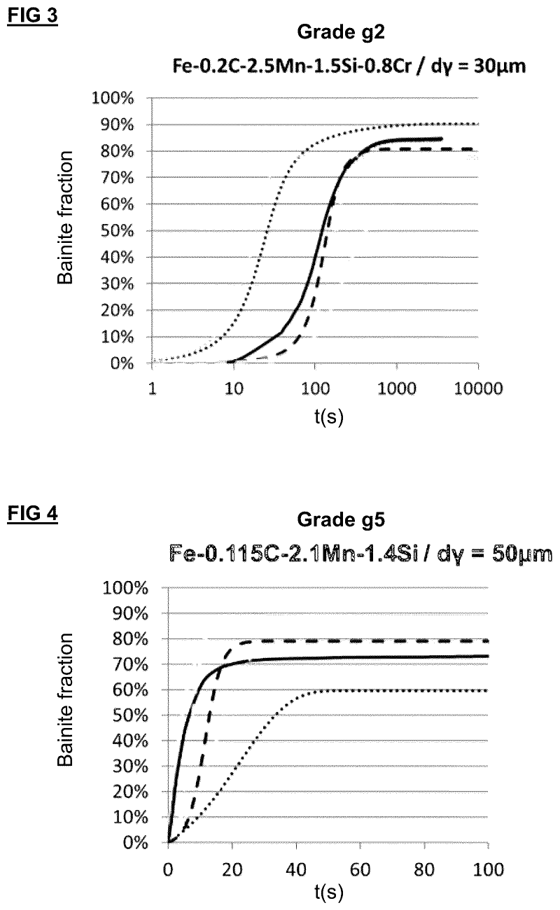

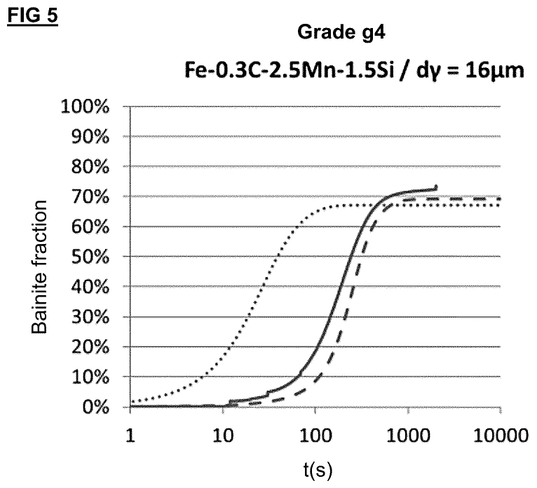

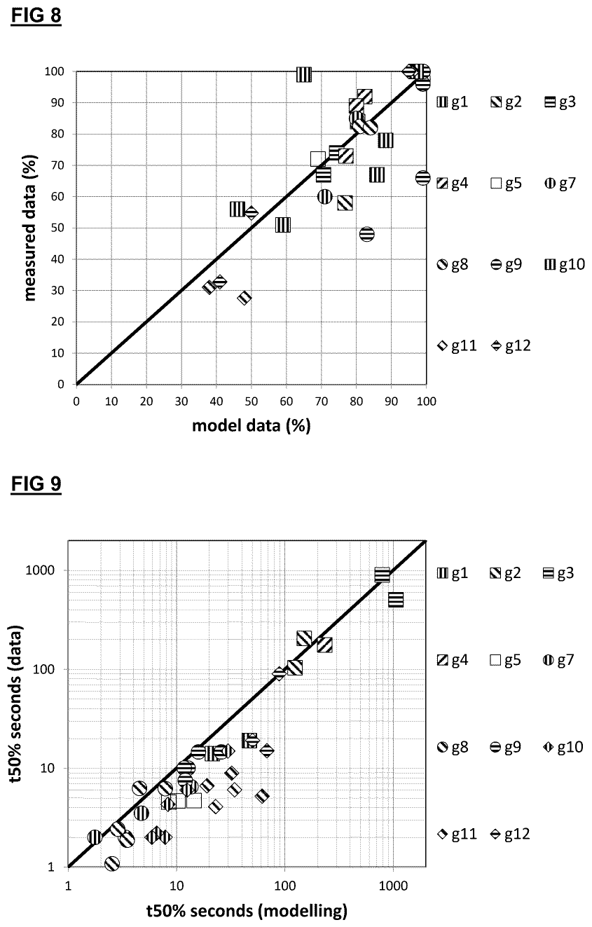

Experimental tests corresponding to steel grades with a high Silicon or Aluminium content, for which carbide precipitation does not occurred (or occurred very slowly during the transformation) are presented in FIGS. 3 to 5, and also in FIGS. 8 and 9.

The transformations considered are isothermal transformations.

The chemical compositions, isothermal temperatures and grain sizes for these grades are gathered in table 1. For each composition, the rest of the composition (in addition to the elements already specified in table 1) is iron and unavoidable impurities resulting from the smelting. The carbon content specified in table 1 is the initial content of Carbon in the austenite, Co. No ferritic transformation occurred in the steel, before these bainitic (isothermal) transformations. The measurements of the bainitic ferrite fraction f_{α} are carried on by synchrotron measurements.

TABLE 1
CFB tested grades

| | | transformation | | | | | | |
|---|---|---|---|---|---|---|---|---|
| Grade | | temperature T | dγ | | Mn | Cr | Si | Al |
| ref (1) | Grade ref (2) | (°C) | (μm) | C % | % | % | % | % |
| g1 | 0.1C 2.5Mn 0.8Cr 1.5Si | 430; 480 | 27 | 0.1 | 2.50 | 0.8 | 1.5 | 0 |
| g2 | 0.2C 2.5Mn 0.8Cr 1.5Si | 380; 430 | 30 | 0.2 | 2.50 | 0.8 | 1.5 | 0 |
| g3 | 0.3C 2.5Mn 0.8Cr 1.5Si | 330; 380 | 32 | 0.3 | 2.50 | 0.8 | 1.5 | 0 |
| g4 | 0.3C 2.5Mn 1.5Si | 400 | 16 | 0.3 | 2.50 | 0 | 1.5 | 0 |
| g5 | 0.1C 2Mn 1.5Si | 500; 550 | 50 | 0.11 | 2.10 | 0 | 1.4 | 0 |
| g6 | 0.25C 2.1Mn 1.5Al | 400; 450; 500; | 56 | 0.25 | 2.07 | 0 | 0 | 1.5 |
| | | 550 | | | | | | |

FIGS. 3, 4 and 5 represent the evolution of the bainitic ferrite fraction f_{α} over time t (in seconds) for the grades g2, g5 and g4 of table 1 respectively, and for respective isothermal transformation temperatures of 380 °C, 550 °C and 400 °C.

In FIGS. 3 to 5, the measured values are represented by the plain-line curve, while the values predicted according to the above-described method are represented by the dashed line. The dotted line corresponds to predictions calculated without the moderation coefficient (that is, with by replacing the moderation coefficient by 1).

FIGS. 3 and 4 illustrate that using the moderation coefficient C_{mod} improves the prediction accuracy regarding the final bainitic ferrite fraction, compared to predictions carried on without taking into account this moderation effect. FIGS. 3 to 5 illustrate also that acceptable, or even good agreements are also obtained for the formation kinetics, using the computation method presented above.

Grades with Cementite (Carbide) Precipitation

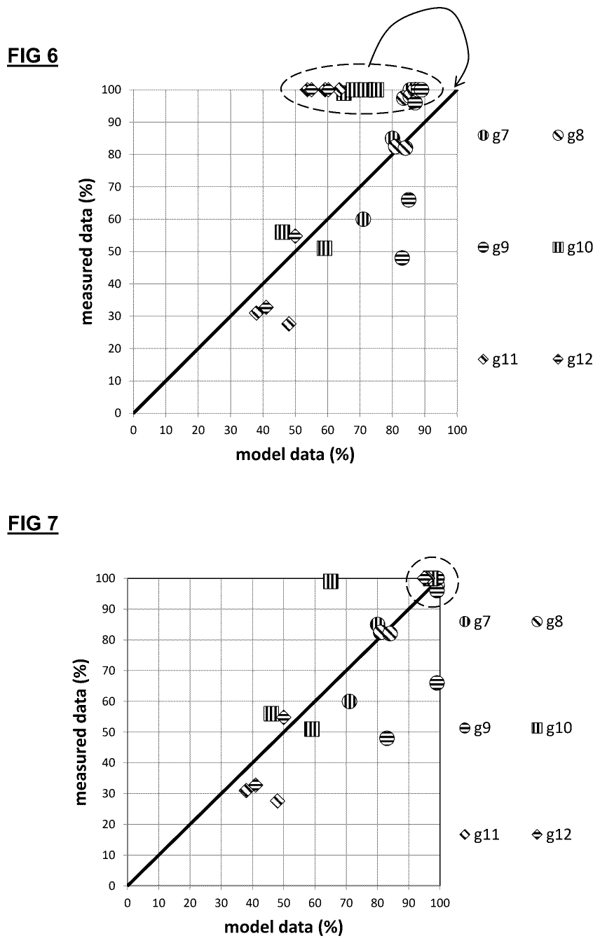

The test results presented in FIGS. 6 and 7 correspond to steel grades with a low Silicon content, of 0.01 wt % or less, for which carbide precipitation occurred during the transformation. The transformations considered are isothermal transformations.

These grades have been homogenized at 1200 °C for 72 hours then air cooled. Ferrite-Pearlite microstructures are then formed at ambient temperature, and a specific treatment (with a double annealing, first at 890 °C for 1 minute, then at 1100 °C for 1 minute) has been applied to erase as much as possible the initial microstructure and form a relatively homogeneous microstructure with big austenite grain size before setting the temperature to the isothermal test temperature to test bainite formation. The grain sizes specified in table 2 are the one employed for the bainitic transformation calculation. Measured grain sizes were of 50 microns in average, comprised between 35 and 60 microns, for these tests.

No ferritic transformation occurred in the steel, before the bainitic (isothermal) transformations presented below.

The chemical compositions, isothermal temperatures and grains size for these grades are gathered in table 2.

TABLE 2
tested grades with possible cementite precipitation

| Grade | Grade ref | Isothermal transformation | dγ | C | Mn | Si |
|---|---|---|---|---|---|---|
| ref (1) | (2) | temperature T (°C) | (μm) | wt % | wt % | wt % |
| g7 | 0.12C 2Mn | 450; 500; 550; 600 | 50 | 0.12 | 2.00 | 0.01 |
| g8 | 0.1C 2Mn | 500; 525; 550; 575; 600 | 50 | 0.09 | 2.01 | 0.01 |
| g9 | 0.1C 3Mn | 425; 450; 475; 500; 525 | 50 | 0.09 | 2.98 | 0.01 |
| g10 | 0.2C 1.5Mn | 500; 525; 550; 575; 600; 625 | 50 | 0.18 | 1.46 | 0.01 |
| g11 | 0.3C 1.5Mn | 500; 525; 550; 575; 600 | 50 | 0.26 | 1.46 | 0.01 |
| g12 | 0.3C 2Mn | 500; 525; 550; 575 | 50 | 0.27 | 1.97 | 0.01 |

FIGS. 6 and 7 represent the total fraction of austenite that has transformed during the transformation (that is f_{γ}(tₒ)−f_{γ}(t_{f}), here equal to 1−f_{γ}(t_{f})), once statis is reached, expressed in %. In FIG. 7, the measured fraction (ordinate axis) is plotted against the fraction computed according to the method presented above (abscissa axis). In FIG. 6, the measured fraction (ordinate, vertical axis) is plotted against a computed fraction (abscissa, horizontal axis) computed according to the method presented above but without taking into account possible cementite formation, that is without step s2. In each figure, the straight line (y=x) corresponds to ideally accurate predictions.

In FIG. 6, for the test results surrounded by a dashed line, austenite is completely transformed into bainitic ferrite and cementite during the transformation, in practice, while the model of FIG. 6 (which does not take cementite formation into account) predicts only a partial transformation of the austenite. On the contrary, the model of FIG. 7 (which corresponds to the computation method presented in detail above) adequately predicts a full austenite transformation, in good agreement with the experiments, for the tests in question, thus illustrating the usefulness of step s2 and of carbon redistribution calculation. More generally, FIG. 7 shows that the method presented above, for determining the amount of bainitic ferrite (and cementite) formed during a transformation leads to final fractions in good agreement with the experiments. For the high Mn content test (grade g9), the agreement is not as good as for the other tests. So, for non-CFB grades, if a high accuracy is requested, the method in question may preferably be employed for Mn contents equal to or lower than 2.5 wt %, or even equal to or lower than 2 wt % (rather than on the whole range x_{Mn}≤3 wt %).

### CFB and Non-CFB Grades Global Results

FIGS. 8 and 9 gather test results, both for the CFB grades g1 to g5 presented above, and for the non-CFB grades g7 to g12.

FIG. 8 represents the total fraction of austenite that has been transformed during the transformation (that is f_{γ}(tₒ)−f_{γ}(t_{f})=1−f_{γ}(t_{f})), once statis reached, expressed in %. Like for FIG. 7, the measured fraction (ordinate axis) is plotted against the fraction computed according to the method presented above (abscissa axis).

FIG. 9 concerns the transformation kinetics. It represents the time t_{50%} to reach 50% of the final fraction of transformed austenite, expressed in seconds. The measured t_{50%} (ordinate axis) is plotted against the value of t_{50%} obtained using the computation method presented above (abscissa axis).

In FIGS. 8 and 9, the straight line (y=x) corresponds to ideally accurate predictions.

FIG. 8 shows that the method presented above, for determining the amount of bainitic ferrite formed during a transformation, leads to final fractions in good agreement with the experiments, for CFB grades as well as for grades with very low Si and Al content.

FIG. 9 shows that the formation kinetics is reasonably well predicted by this method, both for CFB grades and for grades with very low Si and Al content, for response times spanning over three orders of magnitude. Still, it is observed that the predicted kinetics is usually slower than the observed one, specifically for non-CFB grades with a high carbon content (0.3 wt %), namely grades g11 and g12. So, for non-CFB grades, if a high accuracy is requested, the method in question may preferably be employed only for Carbon contents equal to or lower than 0.25 wt %, or even equal to or lower than 0.2 wt % (rather than on the whole range Cₒ≤0.5 wt %).

Test results for grade g6, which contains Aluminum (1.5 wt %) instead of Silicon, have also been compared with the predictions of this computing method, which are in good agreement with the experimental results (8-10% agreement for the final transformed fraction, and about 20-30% for the t_{50%} time).

Phase Fractions and/or Temperature Estimation for a Steel Semi-Product Undergoing the Steel Processing Operation

In the method for determining an amount of bainitic ferrite formed during a steel processing operation, that has been described above, the amount formed at each time step depends on the temperature T of the steel semi-product, which may evolve from one time step to the other (should this temperature be a local temperature at a given position in the steel semi-product, or an average temperature of the steel semi-product). In this regard, the temperature T of the steel, at the different times steps, may be:
either, a known quantity, independent of the phases fractions f_{α}, f_{γ}, f_{c}, and that is acquired by a microstructure calculation module 15′ that determines the temporal evolution of the phases fractions (as shown in FIG. 10); this is the case when a known, predetermined thermal path TP is imposed to the steel semi-product during the steel processing operation, for instance, or when the temperature of the steel semi-product being processed is measured all along the steel processing operation;
or a quantity whose value is computed time step by step, along the steel processing operation, given an initial temperature Ti and heat exchanges between the steel semi-product and its environment (as shown in FIG. 11).

FIG. 10 represents schematically steps executed to determine an amount of bainitic ferrite formed in a steel semi-product during a steel processing operation, according to the method that has been presented above (in the section “bainite formation”).

As represented in FIG. 10, the microstructure calculation module 15′ acquires the chemical composition CC of the steel and a value of the dimension dγ of the austenite grains in the steel. The microstructure calculation module 15′ also acquires data representative of the thermal path TP followed by the steel semi-product during the steel processing operation. These data may specify the temperature imposed to the steel semi-product at each time step, during the processing operation. Alternatively, it may specify an initial temperature, duration, and final temperature for each of the successive phases composing the thermal path TP. The data representative of this thermal path TP may be acquired all at once, before the steel processing operation starts, or continuously, all along the processing operation (when the temperature of the steel semi-product is measured online, several times successively during this processing operation, for instance).

As above mentioned, the microstructure calculation module 15′ executes the set of steps s1, s2 and s3 several times successively, iteratively. At each execution of this set of steps, that is, for each new time step, the value of the temperature T, taken into account to compute the evolution of the phase fractions f_{α}, f_{γ}, f_{c}, is the temperature, at that time step, according to the thermal path TP in question.

FIG. 11 represents schematically steps executed to determine an amount of bainitic ferrite formed in a steel semi-product during a steel processing operation, for another embodiment of the method presented above, in the “bainite formation” section. In the embodiment of FIG. 11, a microstructure calculation module 15 acquires the chemical composition CC of the steel and a value of the dimension dγ of the austenite grains in the steel. The microstructure calculation module 15 also acquires the initial temperature Ti the steel has at the beginning of the steel processing operation (for instance at an input of a steel processing installation where the steel processing operation is achieved, or at an input of a given section of this processing installation).

The microstructure calculation module 15 also acquires data representative of heating or cooling conditions HCC applied to the steel semi-product during the steel processing operation. These data specify operation conditions of heating or cooling devices of the steel processing installation, like the heating power output by heating elements such as radiant tubes, or like the flow rate, speed and/or temperature of a coolant projected by one or more nozzles. Knowing these heating or cooling conditions HCC enables to determine the heat exchanges between the steel semi-product and its environment, during the steel processing operation. The data representative of the heating or cooling conditions HCC may be acquired all at once, before the steel processing operation starts, or continuously, all along the processing operation.

In the embodiment of FIG. 11, the microstructure calculation module 15 determines the temperature T of the steel at the different time steps time step by time step, along the steel processing operation, taking into account:
the initial value of the temperature, To, and
the heat exchanges between the steel semi-product and its environment, determined based on the heating or cooling conditions HCC.

Here, the temporal evolution of the temperature T is determined further taking into account the values, at each time step, of the phase fractions of bainitic ferrite, austenite and cementite, f_{α}, f_{γ} and f_{C}. More specifically, the heat capacity, and possibly the heat conductivity and/or density of the steel are determined based on the current values of f_{α}, f_{γ} and f_{C}. And, at each new time step, the microstructure calculation module 15 execute a step s4 (see FIG. 11), to determine an updated value of the temperature T, based on:
this (continuously updated) heat capacity,
the above-mentioned heat exchanges, and
a value of the temperature T at the previous time-step.

This arrangement enables to take into account the evolution of the heat capacity (and possibly conductivity) of the steel during the steel processing operation, caused by the phase transformations.

During step s4, the updated value of the temperature T may be determined taking also into account the variations of the phases fractions between the previous and next time step, more precisely taking into account the heat absorbed or released due to the variation of these phase fractions. This increases the accuracy of the determination of the temperature T, as the transformation of austenite into bainitic ferrite is exothermal. Regarding heat absorption/release due to cementite formation, it may generally be neglected.

As already mentioned, the microstructure calculation module 15 executes the set of steps s1, s2 and s3 several times successively, iteratively. In the case of FIG. 11, for each new time step, the microstructure calculation module executes:
the set of steps comprising steps s1, s2 and s3,
in parallel, step s4, and
   and then again (iteratively).

Still, the ensemble of steps s1 to s4 could be arranged differently. For instance, step s4 could be executed after step s3.

During the execution of steps s1 and s2, the value of the temperature T taken into account is the value determined during the last execution of step s4 (or, possibly, the initial temperature Ti, for the first execution of steps s1 and s2).

Both in the case of FIG. 10 and in the case of FIG. 11, the microstructure calculation module 15′; 15 may be an electronic device comprising as least a processor and a memory, and being configured, for instance programmed to execute the method for determining f_{α}, f_{γ} and f_{C} (and possibly T), as described above with reference to FIG. 10, or 11. The microstructure calculation module 15′; 15 may also take the form of a computer program or subprogram, that is a group of instructions whose execution on a computer (connected to adequate sensors and/or communication channels) make the computer to execute the method described above with reference to FIG. 10 or 11.

Both in the case of FIG. 10 and FIG. 11, the initial temperature Ti acquired by the electronic device 15′; 15 may, like here, be a temperature that has been measured by a temperature sensor of the steel processing installation (like a pyrometer), and the evolution of the phases fractions (and possibly of the temperature T) is then determined based on this measured temperature. Besides, the data representative of the thermal path TP followed by the steel semi-product (as shown in FIG. 10), or the data representative of the heating or cooling conditions HCC (as shown in FIG. 11) may also be data that are measured by sensors of the steel processing installation (e.g.: steel temperature sensors, or coolant temperature sensors) and/or may be processing setpoints, transmitted to the heating or cooling devices of the installation by an electronic control apparatus of this installation. The method for determining the amount of bainitic ferrite formed during the steel processing operation can then be considered as a non-direct measurement method, for non-directly measuring or predicting the phases fractions f_{α}, f_{γ} and f_{C} and/or for non-directly measuring or predicting the temperature T of the steel semi-product. Indeed, these quantities are then deduced from one or more measurements relative to the steel semi-product and possibly, relative to the process operation.

This non-direct measurement, or prediction, of f_{α}, f_{γ}, f_{C} and/or T may be employed to monitor the steel processing operation (typically in real time), in which case the value of at least one of f_{α}, f_{γ}, f_{C} and T, at least one of the time steps, is transmitted by the microstructure calculation module 15; 15′ to a human-computer interface such as a display screen, to be output (eg: to be displayed to an operator); for instance, final values of f_{α}, f_{γ}, f_{C} (or a final value of T), at the end of the steel processing operation, predicted according to said method, are displayed on a display screen, and are continuously updated each time a new semi-product is input, or each time a new section of a same steel strip enters the steel processing installation.

The non-direct measurement, or prediction, of f_{α}, f_{γ}, f_{C} and/or T mentioned above may also be employed to characterize the steel semi-product having been processed. In this case final values of f_{α}, f_{γ}, f_{C} (or a final value of T), at the end of the steel processing operation, determined according to said method, are output by the microstructure calculation module 15; 15′ and transmitted to a production database in which they are recorded.

The non-direct measurement, or prediction, of f_{α}, f_{γ}, f_{C} and/or T mentioned above may also be employed for controlling the steel processing installation, during the steel processing operation itself, as detailed in the next section “process control”.

### Process Control

One of the possible control methods for controlling the steel processing operation, based on the values of f_{α}, f_{γ}, f_{C} and/or T determined as above explained, is the control method described below, in which a kind of feed-forward control is implemented. In this control method, an electronic control apparatus, including the microstructure calculation module 15; 15′:
acquires the initial temperature Ti,
acquire planned process parameters, intended to be employed during the steel processing operation; said process parameters are either representative of a planned thermal path TP or of planned heating or colling conditions HCC,
determine an estimated final property of the steel semi-product at the end of steel processing operation; said property is either the temperature of the steel semi-product, its phases content (that is the values of f_{α}, f_{γ}, f_{C}), or a mechanical property determined based on its phases content, like its tensile strength or yield strength; said property is determined by computing final values of f_{α}, f_{γ}, f_{C}, or of T as explained above in section “phase fractions and/or temperature estimation”,
compare the estimated final property with a target final property, desired at the end of the processing operation,
adjust the process parameters depending on said comparison,
control the heating or cooling devices of the steel processing installation based on the process parameters thus adjusted.

In an embodiment of this control method, described in more details below with reference to FIG. 12, the property in question is the temperature T of the steel semi-product, which is steel strip 1, and the process parameters in question are cooling conditions, specifying operating conditions of valves 8, 9 that control cooling jets 21 of a Run-Out-Table.

FIG. 12 schematically represents a hot-rolling installation for delivering a hot-rolled steel strip 1, which includes a furnace 2, a rolling mill 3 and a cooling apparatus 4 (namely the Run-Out-Table), for cooling the steel strip 1. The hot-rolling installation comprises also an electronic control apparatus 5 for controlling the cooling apparatus 4.

The strip 1 is for example a steel plate having a thickness comprised between 1 mm and 30 mm.

The steel strip 1, on discharge from the furnace 2 and the rolling mill 3, is moved in a running direction A. In this example, the running direction A of the strip 1 is substantially horizontal. The strip 1 then passes through the cooling apparatus 4, in which the strip is cooled from its initial temperature Ti down to a final temperature (which is for example room temperature, i.e. about 20 °C, but that may also be higher, the end of the cooling occurring once the strip is coiled). The initial temperature Ti is for example substantially equal to the temperature at the end of the rolling of the strip. To is measured using a pyrometer 24 connected to the control apparatus 5. The initial temperature Ti is for example greater than or equal to 600 °C, notably greater than or equal to 800 °C, or even greater than 1000 °C. The strip 1 passes through the cooling apparatus 4 in the running direction A at a running speed which is preferably comprised between 1 m/s and 25 m/s.

In the cooling apparatus 4, at least one first cooling fluid jet 21 is ejected on a top surface of the strip 1, and at least one second cooling fluid jet is ejected on a bottom surface of the strip 1, opposite its top surface. The cooling fluid, also called coolant, is for example water. The cooling apparatus 4 comprises top and bottom valves, 8 and 9, configured for opening or closing the coolant flow in the direction of the steel strip 1.

The electronic control apparatus 5 includes:
the microstructure calculation module 15 of FIG. 11, and
a control module 14, configured for controlling the cooling apparatus 4 according to the temperature T (in particular the final temperature) determined by the microstructure calculation module 15.

The electronic control apparatus 5 is configured for determining the flow for each valve 8, 9, and accordingly for determining which valve 8, 9 needs to be turned on or off. For example, based on a given cooling pattern, the position of the pyrometer 24 and the target temperature, the control module 14 determines which valves 8, 9 need to be turned on or off in order to compensate for initial temperature variation and strip speed variation.

In this example, the microstructure calculation module 15 is configured so that, when determining the evolution of the temperature T and phases content of the strip, the heat exchanges between the strip 1 and its environment are computed according to the formulas presented in paragraphs 55 to 102 of document EP3645182A1, more specifically according to paragraphs 79, 84, 87, 90 and 93. The evolution of the temperature T of the strip is determined as presented above with respect to FIG. 11. So, it may, in particular, be determined as described in paragraphs 55 to 102 of document EP3645182A1, but replacing the phase transformation calculation described in paragraph 101 of EP3645182A1 by the phase transformation calculation above presented in section “bainite formation”, or, possibly, completing the phase transformation calculation described in paragraph 101 of EP3645182A1 with the bainite formation calculation in question.

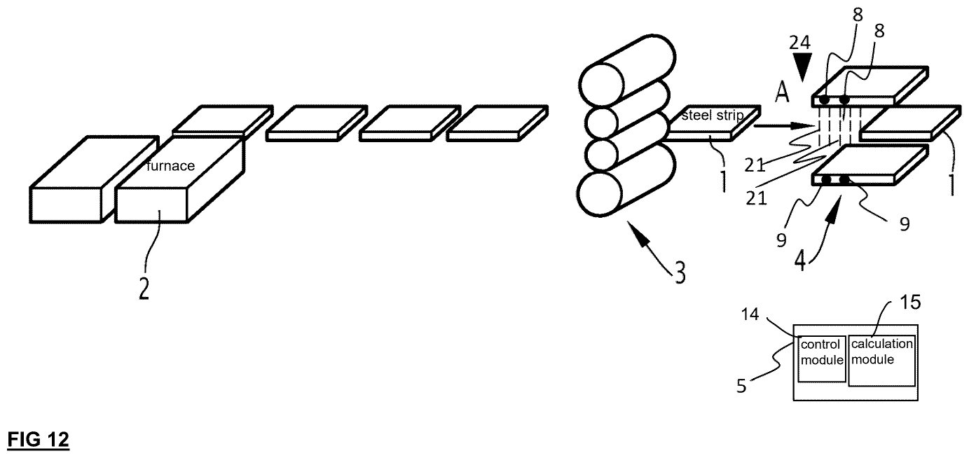

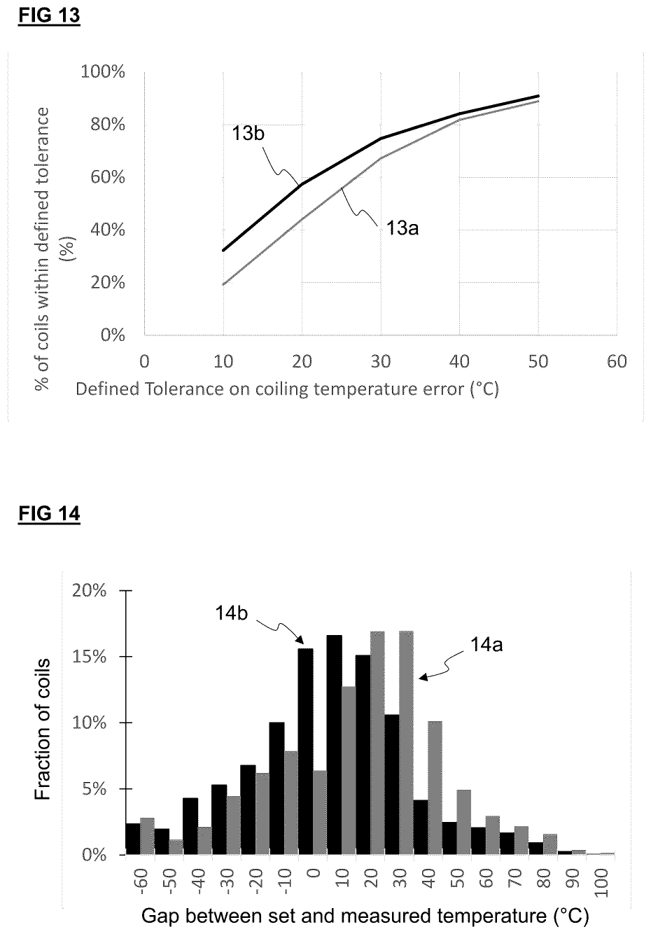

FIGS. 13 and 14 represent production data recorded when operating the cooling apparatus 4, either using the control method that has just been described (curve 13b, histogram 14b), or using the prior art control method described in EP3645182A1 (curve 13a, histogram 14a).

FIG. 13 shows two comparative curves 13a, 13b, regarding the percentage of coils that are provided within a defined tolerance on a coiling temperature error, said defined tolerance being indicated on abscissa axis. Said coiling temperature is the final temperature, at the end of the steel processing operation. Curve 13b illustrates the results of the method according to the invention while curve 13a shows the results of the prior art method (of EP3645182A1). The results with the method according to the invention are better than the ones with the prior art method, since for any value of the defined tolerance indicated on abscissa axis, the percentage of provided coils within said defined tolerance is better with the method according to the invention than with the prior art method.

FIG. 14 shows two comparative histograms 14a, 14b indicating the number of coils that are provided for a respective gap between the target coiling temperature and the measured coiling temperature, said gap being indicated on abscissa axis. Histogram 14b illustrates the results of the method according to the invention while histogram 14a shows the results of the prior art method (of EP3645182A1). The results with the method according to the invention are better than the ones with the prior art method, since histogram 14b is better centred onto zero than histogram 14a, and has values higher than histogram 14a for the small values of the gap (for gap values between −20 and +20 °C).

FIGS. 13 and 14 gather production data obtained coils having each a chemical composition CC within the following ranges (in weight %): Cₒ: [0.065; 0.1], x_{Mn}=[1.3; 1.6], x_{Si}=[0.01; 0.15], x_{Ni}=[0.025; 0.05], x_{Cr}: [0.02; 0.04], x_{Nb}: [0.03; 0.05].

For instance, for one of the coils of FIGS. 13 and 14, the chemical composition was: Cₒ=0.1, x_{Mn}=1.5, x_{Si}=0.09, x_{Al}=0.04, x_{Ni}=0.03, x_{Cr}=0.04, x_{Nb}=0.03, the rest being iron and unavoidable impurities.

Improvements similar, or even higher to those of FIGS. 13 and 14 have also been observed with other grades. For instance, for a grade corresponding to the following ranges: Cₒ: [0.05; 0.06], x_{Mn}=[1.4; 1.5], x_{Si}=[0.2; 0.25], x_{Ni}=[0.025; 0.035], x_{Cr}: [0.3; 0.35], x_{Nb}: [0.025; 0.035].

And also for a grade corresponding to the following ranges: Cₒ: [0.05; 0.07], x_{Mn}=[1.8; 1.9], x_{Si}=[0.02; 0.04], x_{Ni}=[0.025; 0.045], x_{Cr}: [0.02; 0.028], x_{Nb}: [0.06; 0.08], x_{Nb}: [0.06; 0.08], x_{Ti}: [0.1; 0.15].

A method for controlling the coolant jets in a Run-Out-Table, wherein bainite formation is taken into account according to the method presented above (in section “bainite formation”) has just been described.

Still, other steel processing operations can be controlled, according to the invention, using values of f_{α}, f_{γ}, f_{C} and/or T that are determined according to the methods presented above in the section “bainite formation” or in the section “Phase fractions and/or temperature estimation”. For instance, a thermal treatment operation, that follows an annealing, and that includes or precedes a coating operation like a hot-dip coating operation, may be controlled as above explained, in order to obtain a target fraction of bainite at the end of the operation.

### Computer Assisted Design of a Steel Processing Operation

The method for determining an amount of bainitic ferrite formed during a steel processing operation (described in the section “bainite formation”) can also be used for determining, in a Computer Aided Design way, a thermal path to be followed by a steel semi-product in order to obtain a given target property at the end of said processing. Said target property can be:
a target mechanical property, like a tensile or yield strength, deduced from the phase fractions f_{α}, f_{γ}, f_{C},
or the phase fractions f_{α}, f_{γ}, f_{C} themselves.

This thermal path can be determined, for instance, by executing the method of FIG. 10 several times, for different candidate thermal paths, and then selecting the candidate thermal path that leads to a final property, at the end of the steel processing operation, that is the closest to the target property aimed at. Alternatively, a candidate thermal path can be adjusted iteratively, by gradually modifying the thermal path (the method of FIG. 10 being executed at each iteration) until leading to a final property close enough (i.e.: within a given precision range) to the target property. Other schemes for determining (or, in other words optimizing) such a thermal path can also be employed.

Once the thermal path leading to said target property (within a given precision range) is identified, the steel processing operation is executed according to this thermal path.

The determination of such a thermal path, and then control of a steel processing installation accordingly, can be executed by an electronic device or system configured to this end.

What is claimed is:

1-19. (canceled)

20. A method for determining an amount of bainitic ferrite (f_{α}) formed in a steel semi-product during a steel processing operation, the steel semi-product having a chemical composition CC, the method comprising the following steps, executed by an electronic device:
acquiring the chemical composition CC of the steel of the steel semi-product,
s1) determining a bainitic ferrite growth rate, as being proportional to:
a density of bainitic ferrite nucleation centers (n_{nucl,α}),
exp(−Q*/RT) where T is the temperature of the steel, R is the universal gas constant and Q* is an activation energy for an austenite to bainitic ferrite transformation, and to
a moderation coefficient, which approaches zero when a difference ΔG_{γ→α} between a free enthalpy of a bainitic ferrite phase and a free enthalpy of an austenite phase approaches zero, the enthalpy of the austenite phase depending at least on a Carbon content (C^{γ}) in the austenite phase, the value of the Carbon content in the bainitic ferrite phase employed for computing ΔG_{γ→α} being the same as the value of the Carbon content in the austenite phase (C^{γ}),
s2) determining a cementite growth rate, depending at least on the chemical composition CC of the steel and on the Carbon content (C^{γ}) in the austenite phase,
s3) determining values at a next time step for bainitic ferrite, austenite and cementite phases fractions (f_{α}, f_{γ}, f_{C}), based on the bainitic ferrite growth rate and on the cementite growth rate, and determining a value at the next time step for the Carbon content in the austenite phase (C^{γ}) based on the values of said phases fractions (f_{α}, f_{γ}, f_{C}),
the set of steps s1, s2 and s3 being executed several times successively.

21. The method according to claim 20 wherein the moderation coefficient:
is proportional to |ΔG_{γ→α}|/RT when |ΔG_{γ→α}|/RT approaches zero, and
is bound to a constant value when |ΔG_{γ→α}|/RT increases.

22. The method according to claim 21 wherein the moderation coefficient is equal or substantially equal to tanh(|ΔG_{γ→α}|/RT).

23. The method according to claim 20 wherein Q* is equal or substantially equal to Qₒ+K₁·|ΔG_{γ→α}|, and where Q and K₁ depend on the chemical composition CC of the steel but do not vary when the Carbon content (C^{γ}) in the austenite phase evolves, during said processing.

24. The method according to claim 23 wherein Qₒ is equal or substantially equal to A+B·(T−T_{M}), A and B being two constants and T_{M} being the Martensite start temperature for said steel.

25. The method according to claim 20 wherein the density of bainitic ferrite nucleation centers (n_{nucl,α}) is equal or substantially equal to:
an overcooling T_{B}−T, where T_{B} is the Bainite start temperature, multiplied by
a coefficient α^{b}, that depends on the chemical composition CC of the steel and that is proportional to 1/d_{γ}, d_{γ} being a dimension of austenite grains in said steel.

26. The method according to claim 20 wherein the bainitic ferrite growth rate is computed according to equation eqn 1 below
$$\frac{d {f}_{α}}{d t} = v \cdot {n}_{n u c l , α} \cdot {f}_{γ} \cdot ( 1 + λ {f}_{α}^{′} ) \cdot exp ( - {Q}^{*} / RT ) \cdot {C}_{m o d} \qquad ( eqn 1 )$$
where
f_{α} is the fraction of bainitic ferrite in said steel,
C_{mod} is the moderation coefficient,
n_{nucl,α} is the density of bainitic ferrite nucleation centers,
v=kT/h≈10¹³ s⁻¹,
f′_{α} is equal to f_{α}, or possibly to f_{α}+f_{F}, f_{F} being the fraction of ferrite in said steel,
f_{γ} being the fraction austenite in said steel,
and λ is an auto-catalysis coefficient proportional to a dimension d_{γ} of austenite grains.

27. The method according to claim 20 wherein the cementite growth rate is kept equal to zero as long as the Carbon content (C^{γ}) in the austenite phase remains below a Carbon content threshold for cementite precipitation (C^{γ→c}) which depends on the chemical composition CC of the steel.

28. The method according to claim 20 wherein the Carbon content (C^{γ}) in the austenite phase is computed while neglecting a Carbon content in the bainitic ferrite phase (C^{α}) with respect to the Carbon content (C^{γ}) in the austenite phase, and with a carbon content in the cementite phase (C^{c}) that is constant.

29. The method according to claim 20 wherein Carbon, Manganese, Silicon, Aluminum and Chromium contents in said steel, noted respectively as Cₒ, x_{Mn}, x_{Si}, x_{Al} and x_{Cr} and expressed in weight %, are in the following ranges: Cₒ≤0.5; x_{Mn}≤3; x_{Si}≤2; x_{Al}≤1.5; x_{Cr}≤4.

30. A method for monitoring a steel processing operation during which a steel semi-product is processed in a steel processing installation, the method comprising the following steps:
acquiring at least an initial temperature (Ti) of the steel semi-product at a beginning of the steel processing operation, the temperature (T) being measured by a sensor of the steel processing installation, and
determining an amount of bainitic ferrite formed in the steel semi-product during the steel processing operation, according to the method according to claim 20, the method for determining being executed while taking into account that the steel temperature at the beginning of the steel processing operation is the initial temperature measured by said sensor.

31. The method according to claim 30 further comprising
acquiring thermal path data, representative of a thermal path (TP) followed by the steel semi-product during the steel processing operation, and wherein, when determining the amount of bainitic ferrite formed, at each time step, the evolution of the bainitic ferrite phase fraction is determined taking into account a temperature (T) of the steel according to the thermal path.

32. A method according to claim 30 further comprising:
acquiring data representative of heating or cooling conditions (HCC) applied to the steel semi-product (1) during the steel processing operation, and wherein, when determining the amount of bainitic ferrite formed, at each time step, an updated value of the temperature (T) of the steel is determined taking into account said heating or cooling conditions and taking into account the respective phase fractions of bainitic ferrite, austenite and cementite (f_{α}, f_{γ}, f_{C}) in said steel.

33. The method according to claim 32, wherein heating or cooling actuators of the steel processing installation are controlled depending on:
the temperature (T) of the steel expected at an end of the steel processing operation, and depending on
a target temperature, to be reached by the steel semi-product at the end of the steel processing operation.

34. The method according to claim 30, wherein
an estimated final property of the steel semi-product is determined, taking into account the amount of bainitic ferrite formed during the steel processing operation, and
process parameters are adjusted, depending on said estimated final property and depending on a target property for the steel semi-product, to be obtained at the end of the steel processing operation, the process parameters thus adjusted being transmitted to actuators of the steel processing installation for achieving the steel processing operation according to said process parameters.

35. The method according to claim 30, wherein at least one value of the bainitic ferrite fraction determined during said method for determining is output using a human-computer interface, or is recorded in a production database.

36. A method for controlling a steel processing installation, the method comprising the following steps:
determining a thermal path, to be followed by the steel semi-product during the steel processing operation, in order to obtain a given target property for the steel semi-product at an end of said processing, said thermal path being determined taking into account bainitic ferrite formation during said processing, an amount of bainitic ferrite formed during said processing being determined according to the method according to claim 20, and
controlling actuators of the steel processing installation for achieving the steel processing operation according to the thermal path determined.

37. An electronic device comprising at least a processor and a memory, configured for executing the method according claim 20.

38. A computer program, comprising instructions whose execution on a computer make the computer to execute the method according to claim 20.
